(19) United States
(12) Patent Application Publication
(10) Pub. No.: US 2022/0348374 A1
(43) Pub. Date: Nov. 3, 2022

(54) METHOD OF DESIGNING PACKAGING

(71) Applicant: Clinigen Healthcare Limited, Burton-on-Trent (GB)

(72) Inventor: Steven J. Barger, Pottstown, PA (US)

(21) Appl. No.: 17/246,255

(22) Filed: Apr. 30, 2021

(51) Int. Cl. B65B 59/00; B65D 75/04; B65B 5/02; B29C 33/38; B29D 22/00; G06F 30/10

(52) U.S. Cl. CPC B65B 59/001; B65D 75/04; B65B 5/02; B29C 33/3835; B29C 33/3842; B29D 22/003; G06F 30/10; A61M 2209/06; A61M 15/00

(57) ABSTRACT

A method of designing a package configured to securely accommodate one of two or more differently shaped objects. The method comprises: establishing a predetermined orientation for each of the two or more differently shaped objects relative to common axes; identifying dimensionally common points of the two or more differently shaped objects; and developing a design for a package configured to constrain the two or more differently shaped objects at the identified dimensionally common points.

### FIELD

This invention relates to a method of designing a package configured to securely accommodate one of two or more differently shaped objects.

### BACKGROUND OF THE INVENTION

When conducting clinical trials, it is important to ‘blind’ the drug being administered. ‘Blind’ means to ensure that both the clinician and the patient are unaware of whether the drug dosing device contains is a placebo or the trial drug. Where the trial involves the use of a distinctive medical device, such as an inhaler, it can sometimes be possible to tell the placebo from the trial drug by the appearance of the device itself. It is therefore a requirement to conceal the medical device in a way that does not obstruct its operation. Example solutions include adhesive labels to apply to the device so that any markings are difficult to distinguish.

It is an object of the invention to provide a method for designing a package to blind medical devices for clinical trials. More broadly, it is an object of the invention to provide method for designing a package configured to securely accommodate one of two or more differently shaped objects.

### BRIEF SUMMARY OF THE INVENTION

According to embodiments of the invention, there is provided a method of designing a package configured to securely accommodate one of two or more differently shaped objects, the method comprising:
establishing a predetermined orientation for each of the two or more differently shaped objects relative to common axes;
identifying dimensionally common points of the two or more differently shaped objects; and
developing a design for a package configured to constrain the two or more differently shaped objects at the identified dimensionally common points.

The common axes may comprise three axes disposed perpendicular to each other; and wherein identifying dimensionally common points of the two or more differently shaped objects comprises identifying at least one common point of the two or more differently shaped objections along each of the three axes.

The method may further comprise identifying at least two dimensionally common points along each of the three axes.

Developing a design for the package may further comprise establishing primary nodes for contacting the two or more differently shaped objects at the identified dimensionally common points; and secondary nodes for contacting the two or more differently shaped objects at outlying surfaces of the two or more differently shaped objects.

Developing a design for the package may further comprise establishing a surface that is extends between a primary node and a secondary node.

Identifying dimensionally common points of the two or more differently shaped objects may further comprise:
defining a datum point at which each of the three axes intersect;
defining a first plane that lies across two of the three axes;
defining a second plane that lies across another two of the three axes so that the second plane is perpendicular to the first plane;
measuring around the edge of each of the two or more objects in the first plane to establish a silhouette of each object in the first plane;
measuring around the edge of each of the two or more objects in the second plane to establish a silhouette of each object in the second plane;
superimposing the silhouette of each object in the first plane to identify points where the silhouettes intersect in the first plane;
superimposing the silhouette of each object in the second plane to identify points where the silhouettes intersect in the second plane;
establishing said points of intersection as the dimensionally common points.

The method may further comprise manufacturing a package from the design.

The method may further comprise:
developing a mould outline in the first plane that corresponds to the outlying surfaces of the superimposition of the silhouettes in the first plane; and
developing a mould outline in the second plane that corresponds to the outlying surfaces of the superimposition of the silhouettes in the second plane.

The method may further comprise:
projecting the mould outline of the first plane and the mould outline of the second plane to establish a 3D volume enclosed by surfaces of the projected outlines.

According to embodiments of the invention, there is provided a computer program comprising instructions-which, when the program is executed by a computer, cause the computer to carry out the method described above.

The computer program instructions may comprise Boolean uniting, intersecting and subtracting functions.

The method may further comprise making a mould having mould surfaces that correspond to the surfaces of the 3D volume.

The method may further comprise manufacturing a package using a thermoforming, injection moulding process or 3D printing process using the mould having the mould surfaces that correspond to the surfaces of the 3D volume.

Thermoforming or injection moulding may comprise making a moulded receptacle for the two or more differently shaped objects.

The moulded receptacle may comprise a base portion and two side portions that depend from opposite sides of the base portion along respective fold lines, the side portions being configured to fold into facing relation to enclose an object received in the base portion.

The moulded receptacle may comprise an interior space defined by a surface, the surface corresponding to the mould surfaces.

According to embodiments of the invention there is provided a package manufactured according to the method set out above.

According to embodiments of the invention, there is provided a package configured to securely accommodate one of two or more differently shaped objects, wherein the package is configured to constrain the two or more differently shaped objects at dimensionally common points of the two or more differently shaped objects.

The package may further comprise primary nodes for contacting the two or more differently shaped objects at the dimensionally common points; and secondary nodes for contacting the two or more differently shaped objects at outlying surfaces of the two or more differently shaped objects

The package may comprise a moulded receptacle for the two or more differently shaped objects, the moulded receptacle comprising a surface that extends between a primary node and a secondary node.

The moulded receptacle may comprise a base portion and two side portions that depend from opposite sides of the base portion along respective fold lines, the side portions being configured to fold into facing relation to enclose an object received in the base portion.

### BRIEF DESCRIPTION OF THE FIGURES

FIG. 1A shows an inhaler;

FIG. 1B shows another inhaler having a different shape to the inhaler of FIG. 1A;

FIG. 2A shows an open package with the inhaler of FIG. 1A received in the package;

FIG. 2B shows a closed package with the inhaler of FIG. 1A received in the package;

FIG. 2C shows an open package with the inhaler of FIG. 1B received in the package;

FIG. 2D shows a closed package with the inhaler of FIG. 1B received in the package;

FIG. 3A shows a silhouette of a first inhaler in a first plane;

FIG. 3B shows a silhouette of a second inhaler in the first plane;

FIG. 4 shows a superimposition of the silhouettes of the first and second inhalers in the first plane;

FIG. 5A shows a superimposition of the silhouettes of the first and second inhalers in the first plane;

FIG. 5B shows an outline for a mould in the first plane, the mould being adapted to securely accommodate either the first or second inhaler;

FIG. 6A shows how the first inhaler sits within the mould outline in the first plane;

FIG. 6b shows how the second inhaler sits within the mould outline in the first plane;

FIG. 7A shows a silhouette of the first inhaler in a second plane;

FIG. 7B shows a silhouette of the second inhaler in a second plane;

FIG. 8 shows a superimposition of the silhouettes of the first and second inhalers in the second plane;

FIG. 9A shows a superimposition of the silhouettes of the first and second inhalers in the second plane;

FIG. 9B shows an outline for a mould in the second plane, the mould being adapted to securely accommodate either the first or second inhaler;

FIG. 10A shows how the first inhaler sits within the mould outline in the second plane;

FIG. 10b shows how the second inhaler sits within the mould outline in the second plane;

FIG. 11A shows a silhouette first rectangular object in a first plane;

FIG. 11B shows a silhouette a second rectangular object in a first plane;

FIG. 12A shows a superimposition of the silhouettes of the first and second objects in the first plane;

FIG. 12B shows an outline for a mould in the first plane, the mould being adapted to securely accommodate either the first or second object;

FIG. 13 shows the essential steps of a method according to the invention;

FIG. 14A shows a tri-fold moulded pack;

FIG. 14B shows a tri-fold moulded pack with an object nested in the base; and

FIG. 14C shows a tri-fold moulded pack and sleeve.

### DETAILED DESCRIPTION OF THE INVENTION

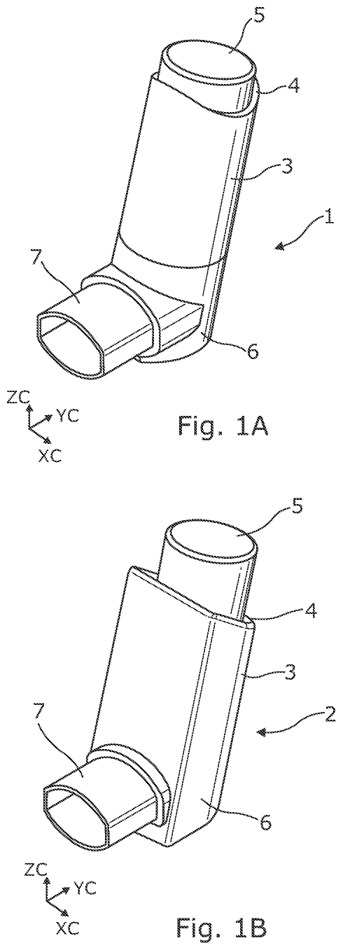

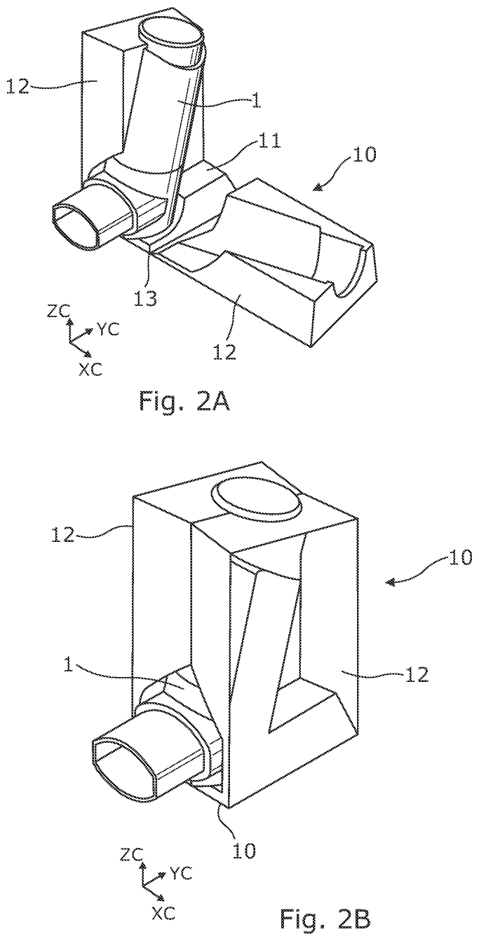

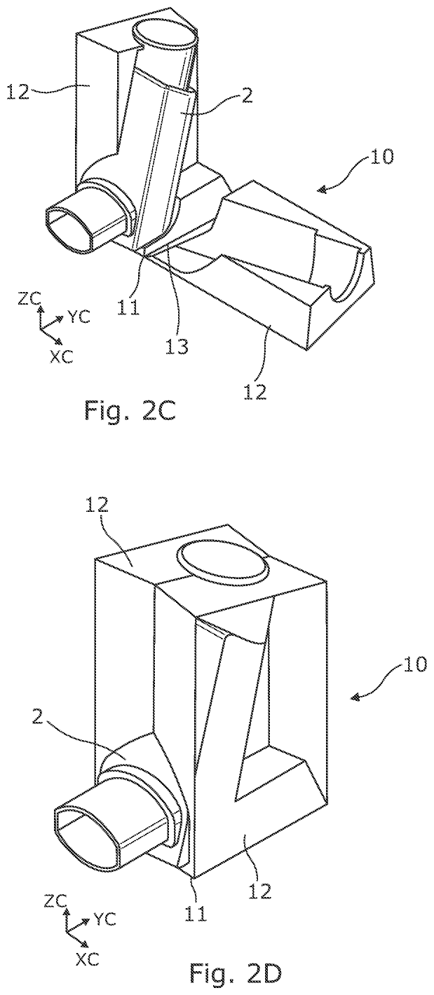

FIGS. 1A and 1B show two differently shaped objects, in this case two differently shaped inhalers 1, 2. Each inhaler 1, 2 comprises a body 3 with an open upper end 4 for a dispensing button 5 and a lower end 6 that communicates with a mouthpiece 7. Although these features are generally common to this type of inhaler, the shape of the body 3 and/or mouthpiece 7 can vary. In the examples shown in FIGS. 1A and 1B, the respective bodies 3 have a different shape. The body 3 of the inhaler 1 of FIG. 1A is substantially cylindrical; while the body 3 of the inhaler 2 of FIG. 1B has a D shaped cross-section. Where such differing inhalers are used in a clinical trial, concealing these differences to those involved in the trial is very important to ensure the reliability of the results. The present invention provides a method of forming a package 10, such as the package 10 shown in FIGS. 2A to 2D, that securely accommodates one of two more differently shaped objects—in this example either of the two inhalers 1, 2 of FIGS. 1B and 1A. It is preferable that the package presents a uniform external appearance to the user. In the example of FIGS. 2A to 2d, this allows the same package 10 to be used for both inhalers 1, 2, increasing the difficulty of discerning one from the other. It is important that the package 10 is configured to fully constrain any of the two or more objects so that they do not rattle around within the package 10. Although the method of the present invention is described primarily with reference to inhalers and the design of a packages for clinical trials, it shall be appreciated that the method described herein applies equally to any differently shaped object where a single package 10 to accommodate either object is required.

In accordance with embodiments of the invention, the method of designing a package 10 to securely accommodate one of two or more differently shaped objects comprises:
establishing a predetermined orientation for each of the two or more differently shaped objects relative to common axes;
identifying dimensionally common points of the two or more differently shaped objects; and
developing a design for a package 10 configured to constrain the two or more differently shaped objects at the identified dimensionally common points.

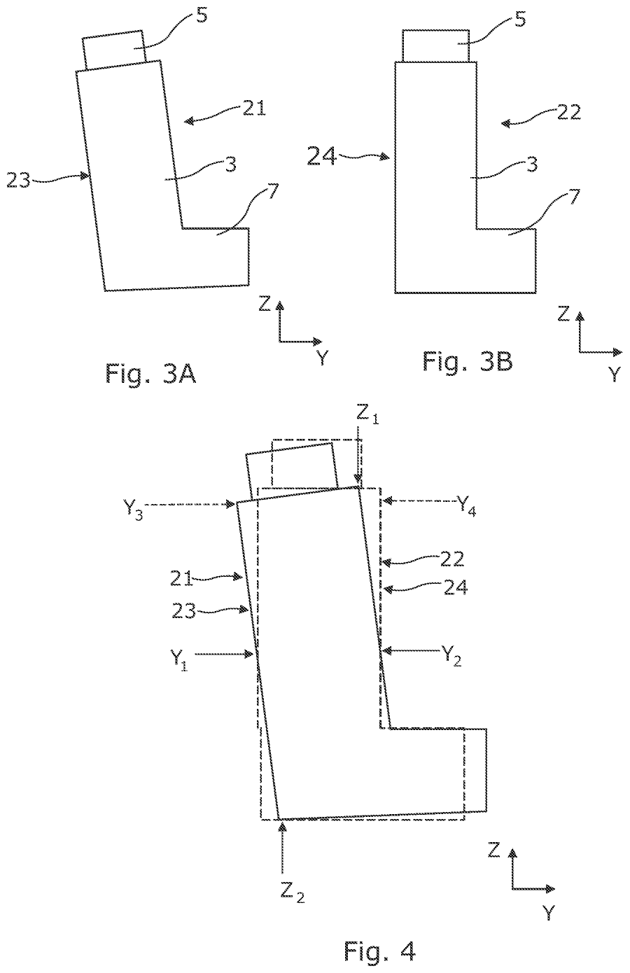

The steps of the method according to a first embodiment are explained in detail with reference to FIGS. 3A to 10B. In this embodiment the method relates to designing and making a package 10 to securely accommodate one of two differently shaped inhalers 21, 22.

In a first step of the method the inhalers 21, 22 are arranged in a predetermined orientation to allow the identification of dimensionally common points. This means that each of the inhalers 21, 22 is provided in a fixed orientation relative to a set of common axes X, Y, Z. This fixed orientation represents the relative position of the inhalers 21, 22 as they will be received in the package 10. For practical purposes, it is advantageous to orient the inhalers so that the mouthpiece 7 and dispensing button 5 are coincident. This means that they will protrude from the package 10 in the same place, allowing common openings to be used—as shown in the example of FIGS. 2A to 2D. However, in the present embodiment, it is not possible to arrange the inhalers 21, 22 to make both the dispensing buttons 5 and mouthpieces 7 exactly coincident, as the body 3 of the first inhaler 21, shown in FIG. 3A, extends at an obtuse angle to the mouthpiece 7, while the body of the second inhaler 22, shown in FIG. 3B, extends perpendicular to the mouthpiece 7. Instead, the inhalers 21, 22 are provided in a fixed orientation that allows the respective mouthpieces 7 and dispensing buttons 5 to be as closely positioned, relative to the common axes, as is possible.

In the conventional way, the axes comprise three axes disposed perpendicular to each other, and are referred to herein as the X axis, Y axis and Z axis. Once the inhalers 21, 22 have been fixed in a predetermined orientation relative to the axes, the next step is to identify dimensionally common points of the inhalers 21, 22 along each of the three axes. One way to achieve this is to measure around the edge of each inhaler in two, perpendicular, planes.

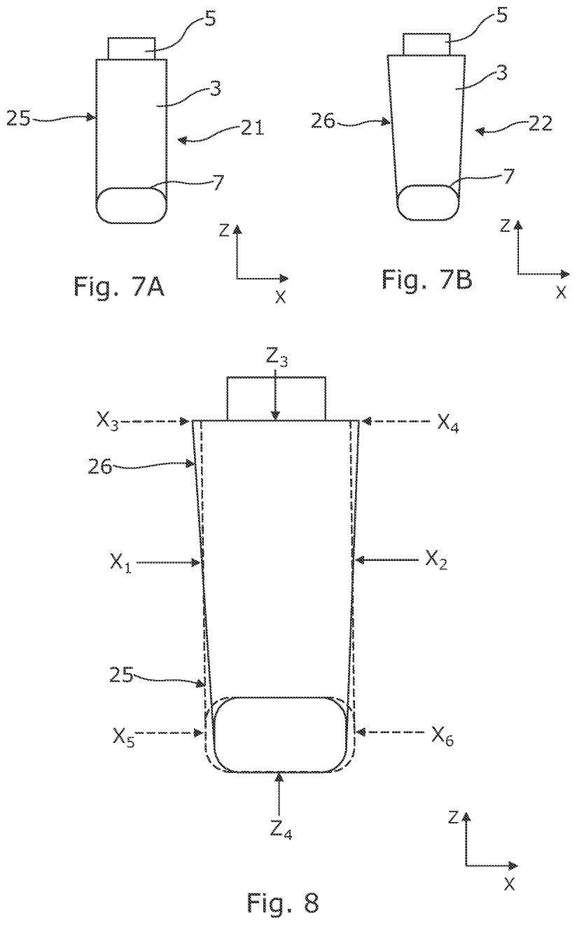

In the illustrated embodiments a first plane is defined that lies across the Z, Y axes, as shown in FIGS. 3A to 6B; and a second plane is defined that lies across the Z, X axes, as shown in FIGS. 7A to 10B. Therefore, the first plane lies perpendicular to the second plane. A datum is defined where each of the three axes intersect. Measurements are then taken around each object in the first and second planes to establish a silhouette of each object in the first and second planes. In practical terms, this means making measurements along the respective axes of the first and second planes to the outlying surfaces of each inhaler 21, 22, thereby establishing a series of coordinates that define the outlying surfaces, as will be explained further below.

With reference to FIG. 3A to 4, in which each inhaler 21, 22 is viewed in the first plane, the inhalers 21, 22 are first established in a predetermined orientation and then fixed in spaced relative to the datum. As explained above, in the case of inhalers, the predetermined orientation aims to align the mouthpieces 7 and dispensing buttons 5 as closely as possible. Next, measurements are taken around the outside of each inhaler 21, 22 from the datum. This means measuring to the external surfaces of each inhaler in turn. Measurements are taken in both the Z axis and Y axis to establish a series of Z, Y coordinates that define the silhouette 23, 24 of the outer surface of each inhaler 21, 22 in the first plane. Time saving techniques may be applied at this step to reduce the number of measurements taken. Simple shapes may require fewer measurements than complex shapes in order to obtain an accurate silhouette. For example, flat surfaces only require the coordinates for the end points of the surface, the surface then being extrapolated by drawing a straight line between these coordinates. Curved surfaces will require more sampling. It is particularly important to obtain the coordinate position of the most outlying points of the silhouette. This means those points that are furthest along the axes. The silhouettes 23, 24 of the respective inhalers 21, 22 in the first plane are shown in FIG. 3A and FIG. 3B, respectively.

As illustrated in FIGS. 7A to 8, this process is then repeated in at least one other plane. In the illustrated embodiment, the process is repeated in the second plane defined by the Z, X axes. Again, measurements are taken of each inhaler 21, 22 in turn to the external surfaces. Measurements in the second plane are made in both the Z axis and X axis to establish a series of Z, X coordinates that define the silhouette 25, 26 of the outer surface of each inhaler 21, 22 in the second plane. The same time saving techniques may be employed when measuring in the second plane as when measuring in the first plane. As in the first plane, it is important to obtain the coordinate position of the most outlying points of the silhouette 25, 26—those points that are furthest along the Z, X axes.

To identify the dimensionally common points the silhouettes 23, 24, 25, 26 of the inhalers 21, 22 are superimposed in each plane. The superimposition of the silhouettes 23, 24 in the first plane is shown in FIG. 4—for clarity, one inhaler 22 is illustrated by a dashed line while the other 21 is illustrated by a solid line. In order to establish the superimposition, the datum point of each silhouette 23, 24 is made coincident. This allows an observer to visually identify the dimensionally common points as they occur where the silhouettes intersect. In FIG. 4, the dimensionally common points are illustrated by the solid arrows Y1, Y2, Z1, Z2. For reasons that will be expanded on, it is important to identify dimensionally common points in each of the three axes. In the first plane, two dimensionally common points are established in the Y axis, denoted by Y1 and Y2; the two dimensionally common points in the Z axis are denoted by Z1 and Z2. The coordinate position of the most outlying points of the superimposition are also established. In FIG. 4, they are shown in Y axis by the dashed arrows, Y3 and Y4.

The superimposition of the silhouettes 25, 26 in the second plane is shown in FIG. 8, with one inhaler 21 illustrated by a dashed line and the other 22 illustrated by a solid line. As in the first plane, the superimposition is established in the second plane by making the datum point for each silhouette 25, 26 coincident. The dimensionally common points are again identifiable as the points where the silhouettes 25, 26 intersect. In FIG. 8, the dimensionally common points are illustrated by the solid arrows X1, X2. As in the other axes, two dimensionally common points are identified, denoted by X1 and X2. The coordinate position of the most outlying points of the superimposition are also established. In FIG. 8, they are shown in X axis by the dashed arrows, X3 to X6.

It shall be appreciated that the dimensionally common points X1, X2, Y1, Y2, Z1, Z2 identified in each axis are defined by the axis in which they prevent movement of the inhalers 21, 22 if the inhalers 21, 22 were to be constrained at those points. Therefore, the identification of two dimensionally common points in each axis allows for the design of a package 10 that will fully constrain the inhaler 21, 22 when the inhaler 21, 22 is received in the package 10. This means that the inhaler 21, 22 will be prevented from rattling around inside the package 10. Because the dimensionally common points X1, X2, Y1, Y2, Z1, Z2 are common to both inhalers 21, 22, either inhaler 21, 22 can be securely accommodated within the package 10. Referring to FIG. 4 the dimensionally common points Y1 and Y2 are chosen to constrain the inhalers in the Y axis, while the dimensionally common points Z1 and Z2 are chosen to constrain the inhalers in the Z axis. Likewise, referring to FIG. 8, the dimensionally common points X1 and X2 are chosen to constrain the inhalers in the X axis. As mentioned, it is important to identify two dimensionally common points in each axis as two opposing constraints are required to prevent movement along that axis. An object properly constrained in each of the X, Y and Z axes is entirely constrained in space.

It is possible to design a package 10 to securely accommodate one or other of the inhalers 21, 22 using the coordinate positions of each of the dimensionally common points X1, X2, Y1, Y2, Z1, Z2. This will be achieved by designing a package having surfaces at these coordinate points so that when either inhaler 21, 22 is received in the package 10, it is fully constrained by these surfaces. The surfaces need to be arranged so that they are at least clear of the most outlying points also identified above. If the surfaces are not clear of these points, they will foul the inhalers 21, 22 when an attempt is made to put them in the package 10.

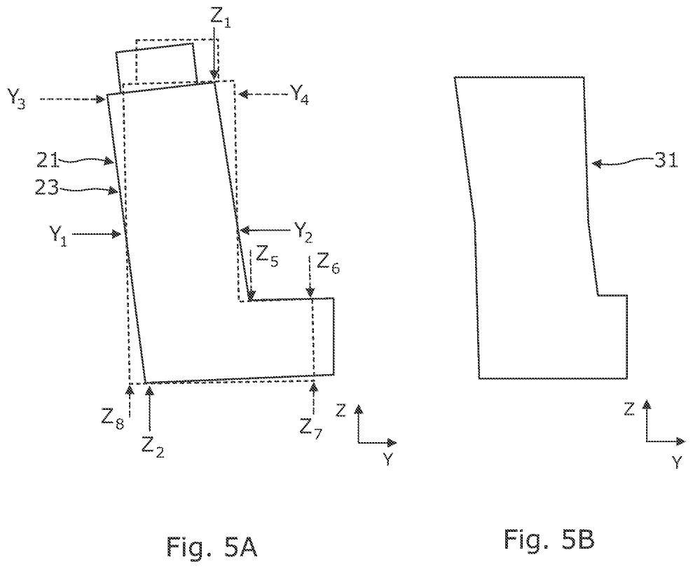

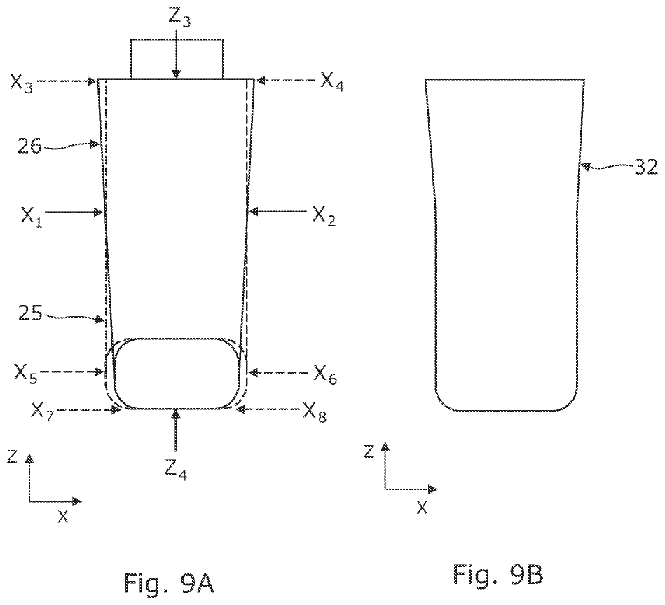

In the embodiment illustrated by FIGS. 5, 5B, 9A and 9B, the coordinate positions of the dimensionally common points X1, X2, Y1, Y2, Z1, Z2 and the coordinate positions of the outlying surfaces can be used to produce an outline for a mould for making the package. FIGS. 5A and 5B illustrate the steps to producing an outline 31 for a mould in the first plane, while FIGS. 9A and 9B illustrate the steps to producing an outline 32 for a mould in the second plane. In the first plane, the dimensionally common points Y1, Y2, Z1, Z2 are established as a primary nodes Y1, Y2, Z1, Z2, while the most outlying points Y3, Y4, Z5, Z6, Z7, Z8 are established as secondary nodes Y3, Y4, Z5, Z6, Z7, Z8. These secondary nodes Y3, Y4, Z5, Z6, Z7, Z8 must be established at vertices of the outlying surfaces. The outline 31 is produced by establishing a straight line between primary nodes Y1, Y2, Z1, Z2 and adjacent secondary nodes Y3, Y4, Z5, Z6, Z7, Z8 so that the outline traces the outlying surfaces of the superimposition of the two inhalers 21, 22. This process is repeated in the second plane, as illustrated by FIGS. 9A and 9B. Primary nodes are established at the dimensionally common points X1, X2 and secondary nodes at the vertices of outlying surfaces X3, X4, X5, X6, X7, X8. In this example, particular care has to be taken around curved surfaces, such as around the lower edge of the mouthpiece. In this case it is important to establish a curve between secondary nodes X5 and X7 and secondary nodes X6 and X8, respectively.

The outlines 31, 32 of FIGS. 5B and 9B are then used to develop a mould. The outline 31 in the first plane represents the cross-sectional shape of the mould in the first plane. Similarly, the outline 32 in the second plane represents the cross-sectional shape of the mould in the second plane. Projecting these sections out of the first plane and second plane, respectively, establishes a 3D volume enclosed by surfaces defined by the projected outlines 31, 32. These surfaces are the mould surfaces. Making a mould to this design allows moulded packaging 10 to be produced that securely accommodates either inhaler 21, 22. Because the surfaces will constrain the inhalers 21, 22 at the primary nodes, the inhalers 21, 22 will be prevented from moving within the package 10.

According to embodiments of the invention, a mould may be made using the surfaces of the above method. The mould may be made according to any conventional process. For example, the surfaces may be CNC machined into steel or aluminium to make the mould tooling. The mould tooling may be used in a thermoforming process or injection moulding process as required to create a package 10 having the package surfaces 40 that correspond to the mould surfaces.

According to embodiments of the invention, the surfaces of the above method may be is directly integrated into a package 10 made using 3D printing technology. For example, the surfaces may be output as an electronic drawing file. A skilled user may then introduce the drawing file into a CAD package to design a package integrating the output surfaces. To allow the design to be 3D printed, the design is encoded as a series of computer readable instructions for a 3D printer. These encoded instructions can then be sent to a 3D printer in the usual way to print the package 10 to the encoded design.

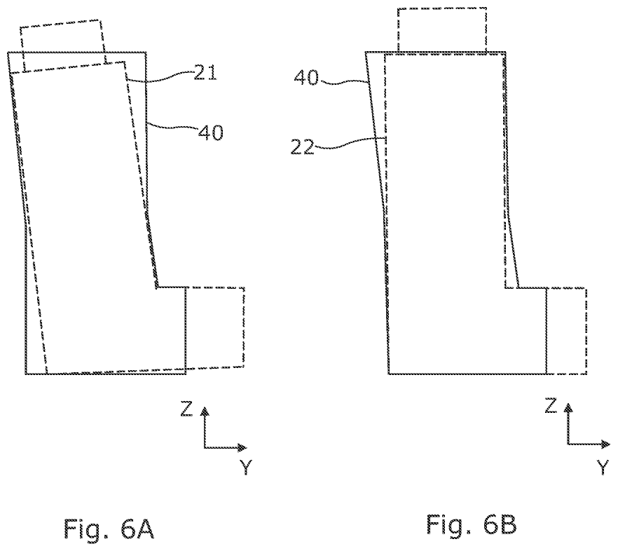

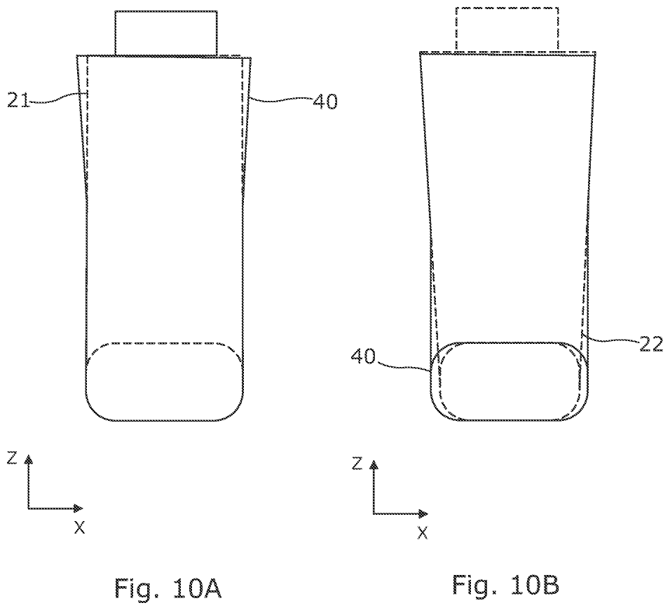

FIGS. 6A and 6B and FIGS. 10A and 10B show how the inhalers 21, 22 of the present embodiment would be accommodated in a package 10 made according to this method in the first and second planes, respectively. In these drawings the solid line represents the outline of the package surfaces 4o, while the dashed line represent the outlying surfaces of the respective inhalers 21, 22. The package surfaces 40 constrain the inhalers 21, 22 at dimensionally common points and provides supporting contact along at least a part of their outlying surfaces.

FIGS. 2A to 2D show a package 10 in accordance with the invention. The package 10 is a tri-fold design, meaning the package comprises a moulded receptacle split into three portions—a base portion 11 and two side portions 12. The side portions 12 depend from opposite sides of the base portion 11 along respective fold lines 13. The side portions 12 are configured to fold into facing relation to enclose an inhaler received in the base portion 11. Each portion comprises surfaces 40 established according to the above method to securely accommodate two different inhalers, in this case the inhalers 1, 2 shown in FIGS. 1A and 1B. Therefore, when the side portions 12 are folded into facing relation, either inhaler of FIG. 1A or FIG. 1B will be securely held within the package 10. It will be further understood that, when the side portions 12 are folded into facing relation, the surfaces 40 of the portions of the package 10 define a 3D volume that corresponds to a 3D volume established in accordance with the method above. In particular, the 3D volume corresponds to a 3D volume established by projecting an outline of the superimposition of the inhalers 1, 2 of FIGS. 1A and 1B in the first and second planes.

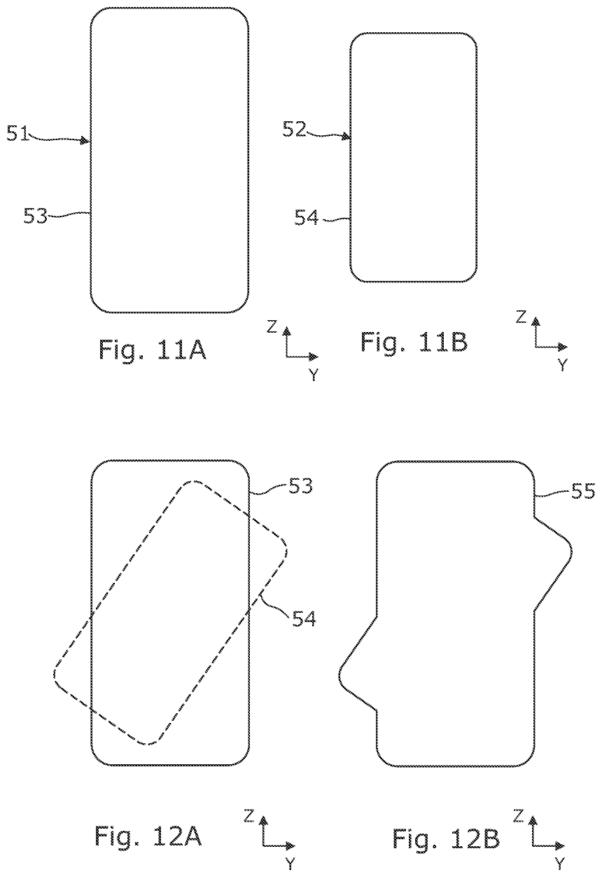

The method described herein provides a solution for the design of a package 10 to accommodate one of two or more differently shaped objects. Although reference is made to inhalers, it will be appreciated that the method may be applied to any two or more objects that can be oriented to provide intersecting surfaces. In another embodiment, shown in FIGS. 11A to 12B, a mould outline is produced in a first plane for two differently sized rectangular objects 51, 52, such as two smartphones. FIG. 11A shows an object 51 with larger overall dimensions than the object 52 illustrated by FIG. 11B. As the outline of the smaller object 52 would fit inside the outline of the larger object 51 when similarly oriented, it is necessary to rotate the objects about the X axis (the axis going into the page) in order to establish intersecting surfaces when the outlines of the objects 51, 52 are superimposed—as shown in FIG. 12A. This illustrates the importance of establishing a predetermined orientation that will enable the identification of dimensionally common points. To develop the mould outline, the steps of the method described above are repeated, briefly:
a predetermined orientation of the two objects 51, 52 is fixed relative to the common axes;
each object is measured around its outer edge in the first plane to establish a silhouette 53, 54 of each object in the first plane;
the silhouettes are superimposed (as shown in FIG. 12A);
a mould outline 55 is developed by tracing around the outlying surfaces of the superimposition of the silhouettes (as shown in FIG. 12B).

These steps may be repeated in the second plane to establish mould sections for the first and second planes. As above, projecting these sections out of the first plane and second plane, respectively, establishes a 3D volume enclosed by surfaces defined by the projected outlines. These will be the mould surfaces for the objects of FIG. 11A and FIG. 11B.

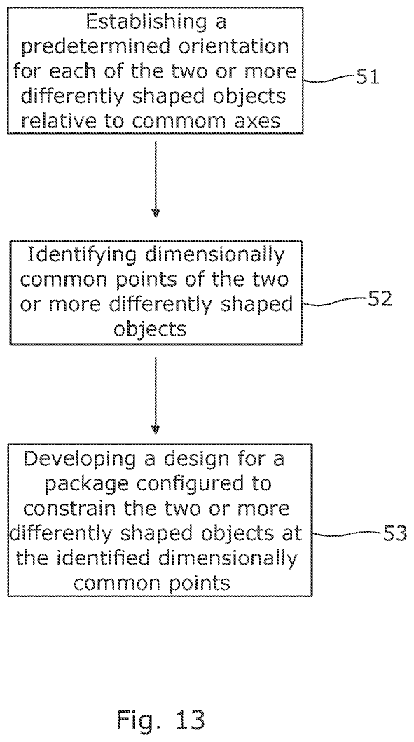

The essential steps of the method according to the invention are shown in FIG. 13 and are the following:

S1) establishing a predetermined orientation for each of the two or more differently shaped objects relative to common axes;

S2) identifying dimensionally common points of the two or more differently shaped objects; and

S3) developing a design for a package configured to constrain the two or more differently shaped objects at the identified dimensionally common points.

These essential steps may be carried out in the physical space, or they may be carried out using software. In one embodiment, the first essential step S1 can be carried out using physical objects and an optical comparator. As the skilled person will appreciate, an optical comparator can be used to take measurements around the outline of the device in any given plane. This can be repeated for each object to establish an outline of each object in its predetermined orientation in at least two perpendicular planes. The outlines may then be superimposed in each plane as described above to establish a mould outline in each plane.

In another embodiment, the package may be designed according to the method described above entirely using software. Instead of the physical objects a direct models of each object may be used to develop surfaces in a 3D coordinate system (x,y,z). The models will be equivalent in scale to each object in 3-dimensional space. By superimposing each model, it is then possible to develop the 3D solid mould surfaces. This may be done using design tools such as Boolean uniting, intersecting etc. of advanced solids modelling CAD software. In one embodiment, a drawing file of each object may be used to develop line drawings in two corresponding elevations. The drawings provide an outline of each object in separate first and second planes. By superimposing each elevation, it is then possible to develop the mould outlines in the first and second planes by drawing around the superimposition in each elevation.

A program may be written to complete the software-based steps automatically. That is, a program may be written that outputs the surfaces of a mould to securely accommodate one of two more objects. By ‘automatically’, it is meant that the program requires only a minimum input from a user. The minimum input will require 3D models of the two or more objects. Other data may also be input into the program, such as a preferred predetermined orientation of the objects. This is useful where certain features of the objects are preferably coincident, such as the mouthpiece and dispensing button of an inhaler. The program is configured to carry out the method above.

In one embodiment, the program carries out the following steps:
inputting the geometry of the differently shaped objects and identifying the 3D surfaces of each object;
defining a predetermined orientation of each object so that the 3D surfaces intersect in at least two perpendicular planes when the 3D objects are superimposed;
superimposing the 3D objects—for example, using Boolean operations—to create a superimposition;
generating a line drawing around the outlying surfaces of the superimposition in each of the two planes;
projecting each line drawing to establish a 3D volume enclosed by surfaces of the projected drawings;
outputting said surfaces as computer readable file.

The output surfaces may be used to drive a CNC machine to make mould tooling in the usual way.

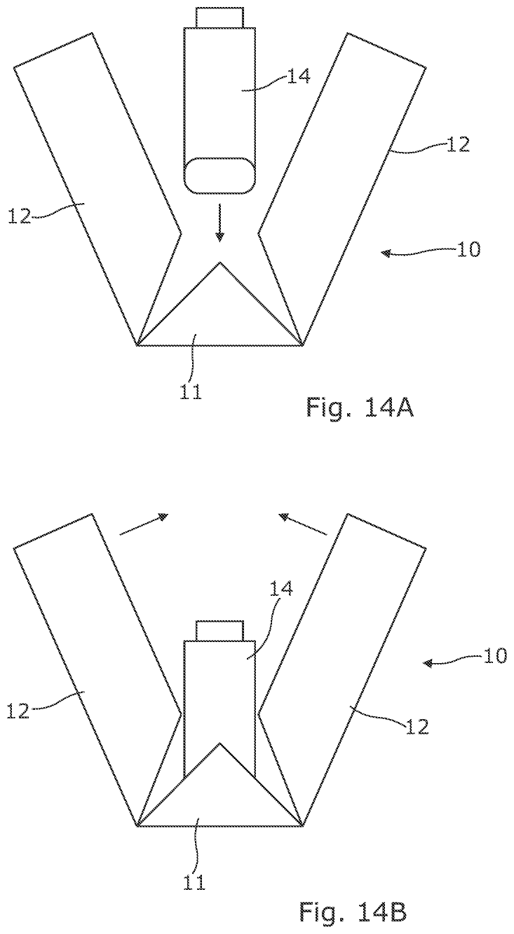

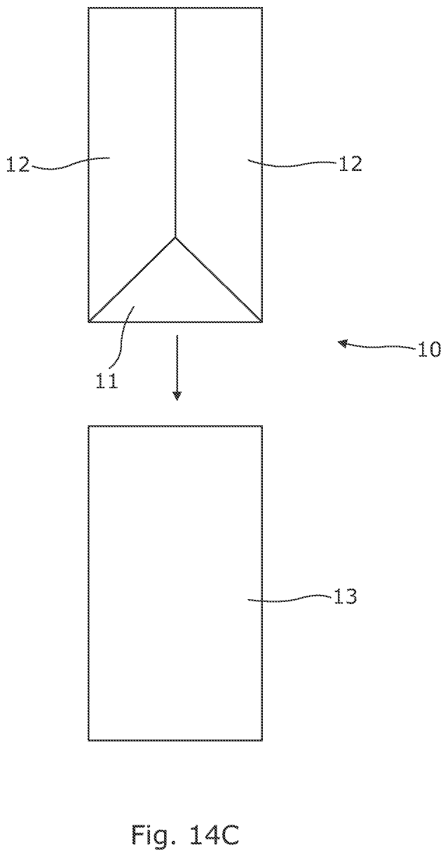

A tri-fold package 10 of the type that can be manufactured using the method disclosed above is shown in FIGS. 14A to 14C. This package is distinct from the package shown in FIGS. 2A to 2C in that the package is configured to completely enclose an object. The package of 2A and 2C instead comprises openings for access to the inhaler dispensing button 5 and mouthpiece 7. This is necessary to allow operation of the inhaler during a clinical trial. In FIGS. 14A to 14C, the object, in this case another inhaler 14, is completely enclosed in the package 10 for transport or storage. As above, the tri-fold package comprises three portions—the base portion 11 and two side portions 12. In order to hold the side portions 12 of the package together, a sleeve 13 is provided to slide over the package 10. The sleeve 13 is one method of closing and, where the package 10 is used for blinding, further concealment. In other embodiments, the sleeve is omitted and the three portions of the package 10 are secured together by other means, such as a tuck-in-tab or flap, an adhesive label or any other method that will be readily appreciated by the skilled person.

It will be appreciated that similar packages 10 may be made in accordance with the method described above to hold any two or more differently shaped objects. The invention is not limited to methods of designing and making packaging for inhalers.

1. A method of designing a package configured to securely accommodate one of two or more differently shaped objects, the method comprising:
establishing a predetermined orientation for each of the two or more differently shaped objects relative to common axes;
identifying dimensionally common points of the two or more differently shaped objects; and
developing a design for a package configured to constrain the two or more differently shaped objects at the identified dimensionally common points.

2. A method according to claim 1, wherein the common axes comprise three axes disposed perpendicular to each other; and wherein identifying dimensionally common points of the two or more differently shaped objects comprises identifying at least one is common point of the two or more differently shaped objects along each of the three axes.

3. A method according to claim 2, further comprising identifying at least two dimensionally common points along each of the three axes.

4. A method according to claim 1, wherein developing a design for the package comprises establishing primary nodes for contacting the two or more differently shaped objects at the identified dimensionally common points; and establishing secondary nodes for contacting the two or more differently shaped objects at outlying surfaces of the two or more differently shaped objects.

5. A method according to claim 4, wherein developing a design for the package further comprises establishing a surface that extends between one of the primary nodes and one of the secondary nodes.

6. A method according to claim 2, wherein identifying dimensionally common points of the two or more differently shaped objects comprises:
defining a datum point at which each of the three axes intersect;
defining a first plane that lies across two of the three axes;
defining a second plane that lies across another two of the three axes so that the second plane is perpendicular to the first plane;
measuring around an edge of each of the two or more objects in the first plane to establish a silhouette of each object in the first plane;
measuring around an edge of each of the two or more objects in the second plane to establish a silhouette of each object in the second plane;
superimposing the silhouette of each object in the first plane to identify points where the silhouettes intersect in the first plane;
superimposing the silhouette of each object in the second plane to identify points where the silhouettes intersect in the second plane;
establishing said points of intersection as the dimensionally common points.

7. A method according to claim 1, further comprising manufacturing a package from the design.

8. A method according to claim 1, wherein the common axes comprise three axes disposed perpendicular to each other; and wherein identifying dimensionally common points of the two or more differently shaped objects comprises identifying at least one common point of the two or more differently shaped objects along each of the three axes, and wherein identifying dimensionally common points of the two or more differently shaped objects comprises:
defining a datum point at which each of the three axes intersect;
defining a first plane that lies across two of the three axes;
defining a second plane that lies across another two of the three axes so that the second plane is perpendicular to the first plane;
measuring around an edge of each of the two or more objects in the first plane to establish a silhouette of each object in the first plane;
measuring around an edge of each of the two or more objects in the second plane to establish a silhouette of each object in the second plane;
superimposing the silhouette of each object in the first plane to identify points where the silhouettes intersect in the first plane;
superimposing the silhouette of each object in the second plane to identify points where the silhouettes intersect in the second plane;
establishing said points of intersection as the dimensionally common points, the method further comprising:
developing a mould outline in the first plane that corresponds to the outlying surfaces of the superimposition of the silhouettes in the first plane; and
developing a mould outline in the second plane that corresponds to the outlying surfaces of the superimposition of the silhouettes in the second plane.

9. A method according to claim 8 further comprising:
projecting the mould outline of the first plane and the mould outline of the second plane to establish a 3D volume enclosed by surfaces of the projected mould outlines.

10. A method according to claim 9, further comprising making a mould having mould surfaces that correspond to the surfaces of the 3D volume.

11. A method according to claim 10, further comprising manufacturing the package using a thermoforming or injection moulding process using the mould having the mould surfaces that correspond to the surfaces of the 3D volume.

12. A method according to claim 11, wherein thermoforming or injection moulding comprises making a moulded receptacle for the two or more differently shaped objects.

13. A method according to claim 12, wherein the moulded receptacle comprises a base portion and two side portions that depend from opposite sides of the base portion along respective fold lines, the side portions being configured to fold into facing relation to enclose an object received in the base portion.

14. A method according to claim 12, wherein the moulded receptacle comprises an interior space defined by a surface, the surface corresponding to the mould surfaces.

15. A package manufactured according to the method of claim 1.

16. A package configured to securely accommodate one of two or more differently shaped objects, wherein the package is configured to constrain the two or more differently shaped objects at dimensionally common points of the two or more differently shaped objects.

17. A package according to claim 16 comprising primary nodes for contacting the two or more differently shaped objects at the dimensionally common points; and secondary nodes for contacting the two or more differently shaped objects at outlying surfaces of the two or more differently shaped objects

18. A package according to claim 17 comprising a moulded receptacle for the two or more differently shaped objects, the moulded receptacle comprising a surface that extends between one of the primary nodes and one of the secondary nodes.

19. A package according to claim 18, wherein the moulded receptacle comprises a base portion and two side portions that depend from opposite sides of the base portion along respective fold lines, the side portions being configured to fold into facing relation to enclose an object received in the base portion.

20. A non-transient computer-readable medium containing instructions for causing a computer to carry out the method of claim 8.
